(19) United States
(12) Patent Application Publication
(10) Pub. No.: US 2025/0050063 A1
(43) Pub. Date: Feb. 13, 2025

(54) CATHETER WITH POLYMER TERMINATION AT TIP SECTION

(71) Applicant: Neuravi Limited, Galway (IE)

(72) Inventors: Karl KEATING, Galway (IE); David VALE, Barna (IE)

(73) Assignee: Neuravi Limited, Galway (IE)

(21) Appl. No.: 18/231,299

(22) Filed: Aug. 8, 2023

(51) Int. Cl. A61M 25/00

(52) U.S. Cl. CPC A61M 25/0068; A61M 25/0009; A61M 25/0045; A61M 25/005

(57) ABSTRACT

A clot capture catheter that includes an elongate tubular shaft and a distal tip section that extends distally from the elongate tubular shaft. The elongate tubular shaft includes a proximal end, a distal end, a lumen, and a tubular support which includes an inner layer comprising a polymer material disposed within the tubular support and an outer layer comprising an elastomeric material disposed over the tubular support. The distal tip section includes a first layer and a third layer comprising an elastomeric material, and a second layer comprising a polymer material.

### FIELD

The present disclosure generally relates to devices and methods for removing blockages from blood vessels during intravascular medical treatments.

### BACKGROUND

Clot retrieval devices are used in mechanical thrombectomy for endovascular intervention, often in cases where patients are suffering from conditions such as acute ischemic stroke AIS, myocardial infarction MI, and pulmonary embolism PE.

Accessing the neurovascular bed in particular is challenging with conventional technology, as the target vessels are small in diameter, remote relative to the site of insertion, and highly tortuous. These catheters are frequently of great length and must follow the configuration of the blood vessels in respect of all branching and windings. Current designs for funnel mouth catheters are often either too large in profile, lack the deliverability and flexibility needed to navigate particularly tortuous vessels, or are not effective at removing a clot when delivered to the target site.

An additional problem that can occur with funnel mouth catheters is delamination at the distal tip section of the catheter. This may lead to functional failures of the device preventing delivery of the catheter to the specified blood clot or a blockage within the lumen that prevent aspiration of the clot.

As a result, there remains a need for improved catheter designs that refine current methods for laminating material onto the distal tip section of the catheter. The presently disclosed designs are aimed at providing an improved catheter tip capable of preventing delamination and methods for fabricating such a catheter.

### SUMMARY

In some examples, a clot capture catheter is disclosed for removing a clot from a blood vessel. The clot capture catheter can include an elongate tubular shaft and a distal tip section that extends distally from the elongate tubular shaft. The elongate tubular shaft can include a proximal end, a distal end, a lumen, and a tubular support which can include an inner layer comprising a polymer material disposed within the tubular support and an outer layer comprising an elastomeric material disposed over the tubular support. The distal tip section can include a first layer and a third layer having an elastomeric material, and a second layer having a polymer material.

In some examples, the first layer and the third layer can be joined at the distal end of the distal tip section.

In some examples, the tubular support of the clot capture catheter can include a braid.

In some examples, the first layer, third layer, and outer layer can include 2 different elastomeric materials or the same elastomeric material.

In some examples, the first layer comprises a proximal terminate within the distal tip section.

In some examples, the distal tip section can include a funnel shape such that a diameter of a distal end of the tip section is greater than a diameter of a proximal end of the tip section.

In some examples, the inner surface of the inner layer and second layer can be chemically etched.

In some examples, the outer surface and at least a distal portion of the inner surface of the second layer is chemically etched.

In some examples, the first layer can include a thickness of at least 0.0005″ and the third layer can include a thickness of at least 0.003″.

In some examples, the third layer of the distal tip section and the outer layer of the elongate tubular shaft are contiguous.

In some examples, a method of manufacturing a clot capture catheter is disclosed. The method can include placing an inner layer having an elastomeric material over a tapered mandrel, placing a tubular support over the inner layer leaving an exposed outer surface at a distal end of inner layer, flaring a distal end of the inner layer into a funnel shape, laminating an outer layer having elastomeric material over at least a portion of the tubular support and up to a proximal end of the funnel shape, replacing the tapered mandrel with a mandrel pre-laminated with a first layer of elastomeric material, and laminating a third layer of elastomeric material over the exposed outer surface of the inner layer.

In some examples, the method can further include terminating a distal end of the distal tip section by trimming excess elastomeric material.

In some examples, the method can further include chemically etching the outer surface of the first layer and the outer surface of the outer layer.

In some examples, the method can include flaring the tapered mandrel to approximately a millimeter.

In some examples, the method can further include laminating the second layer up to a distal end of the tubular support.

In some examples, the method can include a second and third layer that has a thickness of at least 0.003″.

In some examples, the method can further include laminating a fourth layer of polymer material over some or all of the distal tip of the catheter.

In some examples, a method of manufacture for a clot capture catheter is disclosed. The method can include placing a first layer having polymer material over a mandrel, placing a tubular support over the first layer leaving a distal portion of the first layer exposed, laminating a second layer having elastomeric material over at least a portion of the tubular support, inverting the distal portion of the first layer proximally over an outer surface of the second layer and laminating the layers together, removing the mandrel, and flaring a distal tip of catheter using a tapered mandrel.

In some examples, the method can further include chemically etching an outer surface of the first layer.

In some examples, the method can include inverting the distal portion of the first layer such that both an inner and outer surface of the second layer is in contact with the outer surface of the first layer.

In some examples, the method can include laminating the second layer up to a distal end of the tubular support, leaving the distal portion of the first layer exposed.

In some examples, the method can include a second layer that has a thickness of at least 0.003″.

In some examples, the method can include flaring the distal tip of the catheter approximately a millimeter using the tapered mandrel.

Other aspects and features of the present disclosure will become apparent to those skilled in the pertinent art, upon reviewing the following detailed description in conjunction with the accompanying figures.

### BRIEF DESCRIPTION OF THE DRAWINGS

The above and further aspects of this disclosure are further discussed with the following description of the accompanying drawings, in which like numerals indicate like structural elements and features in various figures. The drawings are not necessarily to scale, emphasis instead being placed upon illustrating principles of the disclosure. The figures depict one or more implementations of the inventive devices, by way of example only, not by way of limitation. It is expected that those of skill in the art can conceive of and combining elements from multiple figures to better suit the needs of the user.

FIG. 1 is a view of a blood vessel and a clot and a clot capture catheter disposed therein according to this disclosure.

FIG. 2 is a perspective view of a clot capture catheter according to this disclosure.

FIG. 3A is a cross-sectional view illustrating a step of laminating an outer layer of elastomeric material over an inner layer of polymer material in the method of manufacture for a clot capture catheter according to this disclosure.

FIG. 3B is a cross-sectional view illustrating a step of laminating a third layer over an exposed outer surface of the second layer in the method of manufacture for a clot capture catheter according to this disclosure.

FIG. 3C is a cross-sectional view illustrating the result of terminating the distal end of the distal tip section by trimming excess elastomeric material in the method of manufacture for a clot capture catheter according to this disclosure.

FIG. 4A is a flow diagram illustrating a method of manufacture for a clot capture catheter according to this disclosure.

FIG. 4B is a flow diagram illustrating an alternate method of manufacture for a clot capture catheter according to this disclosure.

FIG. 5A is a side view illustrating a step of placing a tubular support over the first layer in the method of manufacture for a clot capture catheter according to this disclosure.

FIG. 5B is a side view illustrating a step of laminating a second layer over the tubular support in the method of manufacture for a clot capture catheter according to this disclosure.

FIG. 5C is a side view illustrating a step of inverting a first layer over an outer surface of a second layer in the method of manufacture for a clot capture catheter according to this disclosure.

FIG. 5D is a side view illustrating a step of flaring a distal tip of a catheter in the method of manufacture for a clot capture catheter according to this disclosure.

FIG. 6 is a flow diagram illustrating an alternate method of manufacture for a clot capture catheter according to this disclosure.

### DETAILED DESCRIPTION

The following detailed description is merely exemplary in nature and is not intended to limit the disclosure or the application and uses of the disclosure. Although the description of the disclosure is in many cases in the context of treatment of intracranial arteries, the disclosure may also be used in other body passageways as previously described.

The terms “distal” or “proximal” are used in the following description with respect to a position or direction relative to the treating physician. “Distal” or “distally” are a position distant from or in a direction away from the physician. “Proximal” or “proximally” or “proximate” are a position near or in a direction toward the physician.

As discussed herein, a “patient” or “subject” can be a human or any animal. It should be appreciated that an animal can be a variety of any applicable type, including, but not limited to, mammal, veterinarian animal, livestock animal or pet-type animal, etc. As an example, the animal can be a laboratory animal specifically selected to have certain characteristics similar to a human e.g., rat, dog, pig, monkey, or the like.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g. “about 90%” may refer to the range of values from 71% to 99%.

When used herein, the terms “tubular” and “tube” are to be construed broadly and are not limited to a structure that is a right cylinder or strictly circumferential in cross-section or of a uniform cross-section throughout its length. For example, the tubular structure or system is generally illustrated as a substantially right cylindrical structure. However, the tubular system may have a tapered or curved outer surface without departing from the scope of the present invention.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

It must also be noted that, as used in the specification and the appended claims, the singular forms “a,” “an,” and “the” include plural referents unless the context clearly dictates otherwise. Ranges can be expressed herein as from “about” or “approximately” one particular value and/or to “about” or “approximately” another particular value. When such a range is expressed, other exemplary examples include from the one particular value and/or to the other particular value.

Accessing cerebral, coronary and pulmonary vessels involves the use of a number of commercially available products and conventional procedural steps. Access products such as guidewires, guide catheters, angiographic catheters and microcatheters are described elsewhere and are regularly used in catheter lab procedures. It is assumed in the descriptions below that these products and methods are employed in conjunction with the device and methods of this disclosure and do not need to be described in detail.

A common theme across many of the disclosed designs is a clot capture catheter that includes an elongate tubular shaft and a distal tip section that extends distally from the elongate tubular shaft. The elongate tubular shaft includes a proximal end, a distal end, a lumen, and a tubular support which includes an inner liner disposed within the tubular support and an outer liner disposed over the tubular support. The distal tip section includes an inner layer and an outer layer having an elastomeric material, and a middle layer having a polymer material.

The tubular support of the designs disclosed are desirably made from a material capable of recovering its shape automatically once released from a highly strained delivery configuration. A superelastic material such as Nitinol or an alloy of similar properties is particularly suitable. The material can be in many forms such as wire or strip or sheet or tube. A particularly suitable manufacturing process is to laser cut a Nitinol tube and then heat set and electropolish the resultant structure to create a framework of struts and connecting elements. This framework can be any of a huge range of shapes as disclosed herein and may be rendered visible under fluoroscopy through the addition of alloying elements such as platinum or through a variety of other coatings or marker bands.

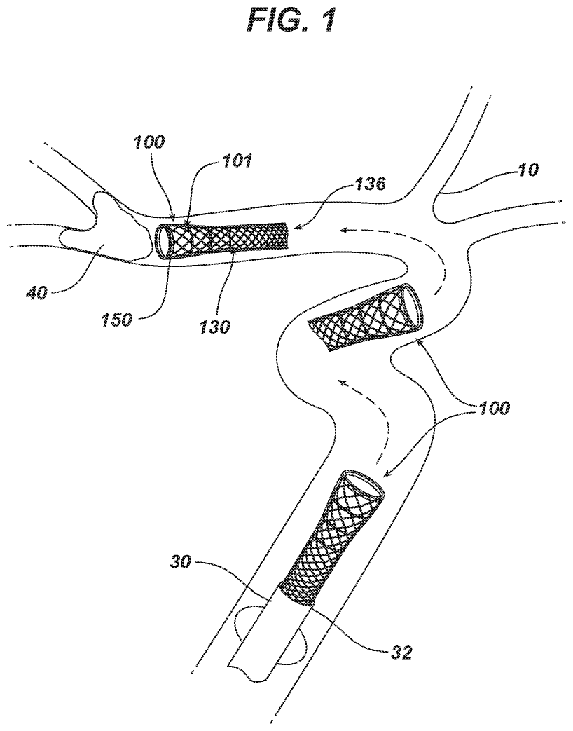

FIG. 1 is an illustration of a possible sequence for approaching an occlusive clot 40 using a clot capture catheter 100 of the designs disclosed herein. The clot 40 can be approached with the catheter 100 collapsed within a guide sheath 30 or other outer catheter for delivery. When the vasculature 10 becomes too narrow and/or tortuous for further distal navigation with the guide sheath 30, the catheter 100 can be deployed for further independent travel distally from the distal end 32 of the guide sheath. The catheter 100 can be highly flexible such that it is capable of navigating vasculature 10 or other tortuous regions of the neurovascular to reach an occlusive clot.

In some examples, the clot capture catheter assembly includes a catheter 100, an elongate tubular shaft 130, a distal tip section 101, a proximal end 136, and a guide sheath 30 that can be configured to navigate vasculature 10. As the catheter 100 approaches the clot 40, the distal funnel portion 150 can expand to approximately abut the walls of the vasculature 10, as described further below.

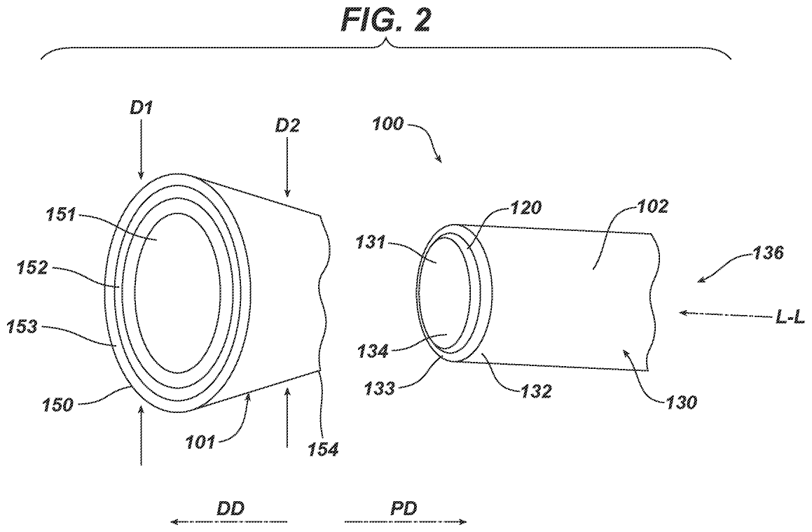

FIG. 2 is a side view of clot capture catheter 100. The catheter 100 can include an elongate tubular shaft 130, a distal end 133, a lumen 134, and a tubular support 120. The tubular shaft can include an inner layer 131 disposed within the tubular support 120 that can include a polymer material and an outer layer 132 disposed over the tubular support 120 that can include an elastomeric material. The distal tip section can include a first layer 151 disposed on only the distal end of the distal tip section and a third layer 153 that can include an elastomeric material. These layers can be reflowed/melted together to form a chemical bond. This chemical bond can be formed through openings in the tubular support 120 in order to form a strong adhesion. The distal tip section can also include a second layer 152 that can include a polymer material. In some examples, the tubular support 120 can include a braid. In some examples the distal tip section 101 can have a funnel shape. In some examples, the first 151 and third 153 layers extend distally past the distal end 121 of the tubular support 120. These layers can be reflowed/melted together to form a chemical bond at the distal end of the distal tip section 101.

In some examples, the first layer 151 comprises a proximal terminate within the distal tip section 101.

In some examples, the distal tip section 101 extends distally from the elongate tubular shaft 130 and the tip 101 can include the first layer 151 and third layer 153 being joined at a distal end 150 of the distal tip section 101. Further, in some examples, the shaft 130 can include an outer layer 132 which can include an elastomeric material and an inner layer 131 can include a polymer material.

In some examples, the first layer 151 can include an elastomeric material, the third layer 153 can include an elastomeric material, and the second layer 152 can include a polymer material.

In some examples, the first layer 151, third layer 153, and outer layer 132 can include a combination of two separate elastomeric materials such as NEUsoft™, Chronoprene®, Pebax®, or Engage™. The material should be soft and elastomeric so that it is atraumatic to the vessel wall and allow a funnel tip to collapse through a guide sheath and expand in a blood vessel.

In some examples, the second layer 152 and inner layer 131 can include a polymer material such as PTFE, wrapped PTFE, WEPL, ePTFE, wrapped ePTFE, HDPE, Nylon, or PEEK. The material should have low friction and allow the passage of other devices.

In some examples, the first layer 151 can have a thickness of at least 0.0005″ and the third layer 153 can have a thickness of at least 0.003″.

In some examples, the third layer 153 of the distal tip section 101 and the outer layer 132 of the elongate tubular shaft 130 are contiguous.

In some examples, the distal tip section 101 can have a funnel shape such that a diameter D1 of a distal end of the distal tip section 150 is greater than a diameter D2 of a proximal end of the tip section 154.

In some examples, the outer surfaces of the second layer 152 and the inner layer 131 can be chemically etched. Further, in some examples, at least a distal portion of the inner surface of the second layer 152 can be chemically etched where it comes in contact with the outer surface of the first layer 151.

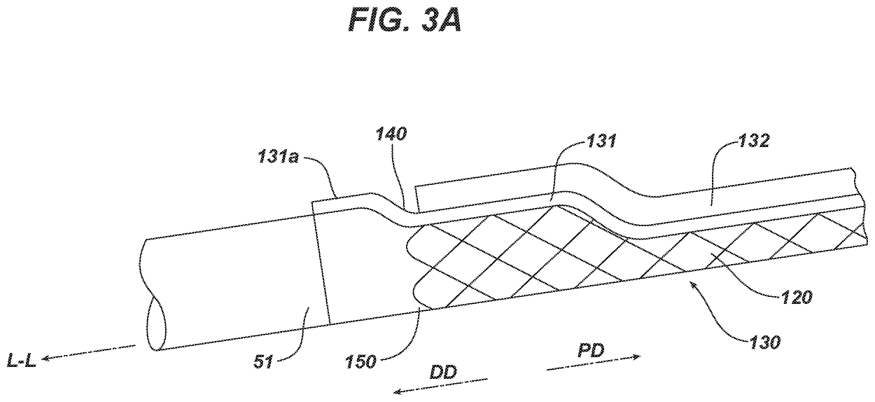

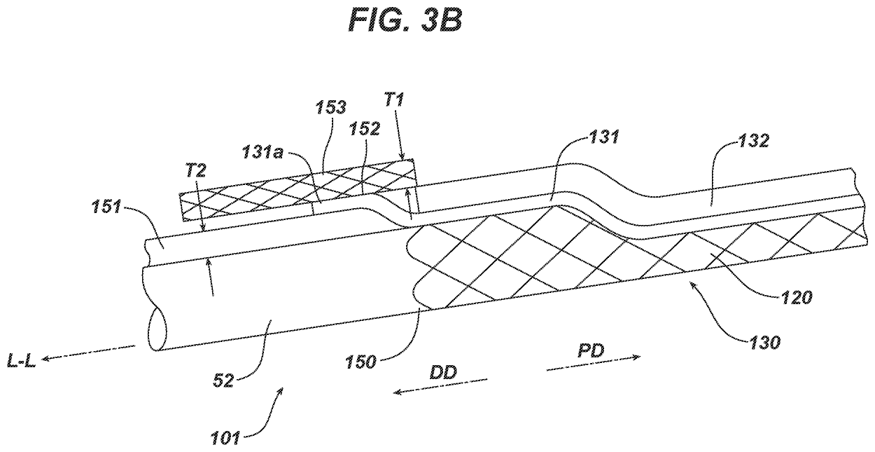

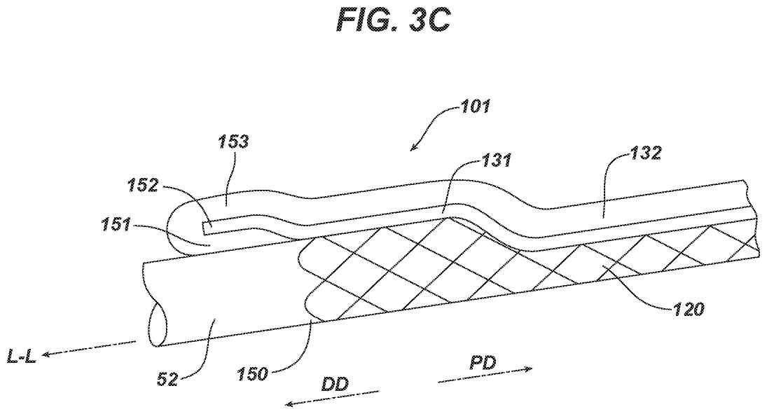

FIG. 3A is a cross-sectional view illustrating a step of laminating an outer layer 132 of elastomeric material over an inner layer 131 of polymer. In some examples, the outer layer 132 is laminated up to a distal end of the tubular support 120.

In some examples, the step shown in FIG. 3A can include a tubular shaft 130 and a distal tip section 101. The distal tip section 101 can include a second layer 152 of polymer. The tubular shaft can include an inner layer 131 of polymer material laminated up to a distal end 150 of distal tip section 101, a tubular support 120 loaded over the inner layer 131, and an outer layer 132 of elastomeric material laminated up to a proximal end of the funnel shape 140, leaving a distal end 131a of the second layer 152 exposed.

FIG. 3B is a cross-sectional view illustrating a step of laminating a third layer 153 of elastomeric material over an exposed outer surface 131a of the second layer 152 of polymer material.

In some examples, the catheter assembly in FIG. 3B can include a tubular shaft 130 and a distal tip section 101. The tubular shaft can include an inner layer 131 of polymer material laminated up to a distal end 150 of distal tip section 101, a tubular support 120 loaded over the inner layer 131, and an outer layer 132 of elastomeric material laminated up to a proximal end of the funnel shape 140. The distal tip section can include a mandrel 52, a first layer 151 of elastomeric material laminated over mandrel 52, a second layer 152 of polymer material, and a third layer of elastomeric material 153 laminated over the exposed area 131a of the second layer 152.

In some examples, the first layer 151 can have a thickness T2 and the third layer 153 can have a thickness T1 that is greater than thickness T2.

FIG. 3C is a cross-sectional view illustrating the result of terminating the distal end of the distal tip section 150 by trimming excess elastomeric material.

In some examples, the catheter assembly in FIG. 3C can include a tubular shaft 130 and a distal tip section 101. The tubular shaft 130 can include an inner layer 131 of polymer material, a tubular support loaded over the inner layer 131, and an outer layer 132 of elastomeric material over the tubular support 120. The distal tip section 101 can include a first layer 151 of elastomeric material, a second layer 152 of polymer material, and a third layer 153 of elastomeric material.

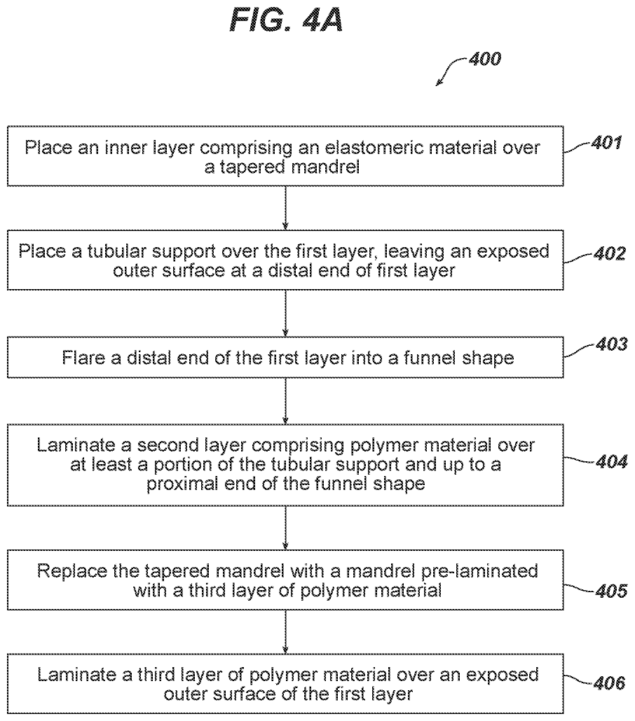

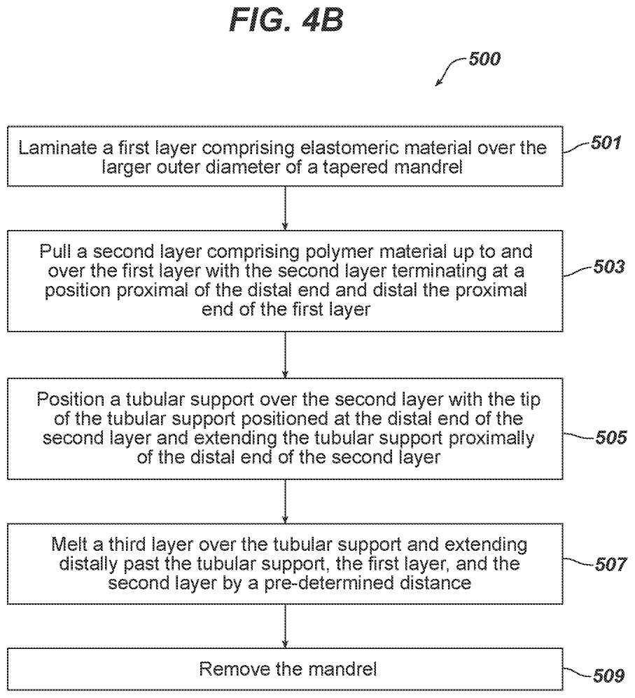

FIG. 4A shows a method 400 of manufacture for a clot capture catheter as disclosed herein. The method steps in FIG. 4A can be implemented by any of the example means described herein or by similar means, as will be appreciated.

At block 401, the method 400 can include placing an inner layer comprising an elastomeric material 131 over a tapered mandrel 51. The tapered mandrel can be configured similarly to an example tapered mandrel 51 disclosed herein, variations thereof, and alternatives thereto as understood by a person skilled in the pertinent art.

In some examples, the method 400 can include chemically etching the inner layer 131. The chemical etching can be used to assist in the bonding between the subsequent layers.

At block 402, the method 400 can include placing a tubular support 120 over the inner layer 131, leaving an exposed outer surface 131a at a distal end of inner layer 131. The tubular support can be configured similarly to an example tubular support 120 disclosed herein, variations thereof, and alternatives thereto as understood by a person skilled in the pertinent art.

At block 403, the method 400 can include flaring a distal end of the inner layer 131 into a funnel shape. In some examples, the tapered mandrel can be flared approximately a millimeter.

At block 404, the method 400 can include laminating an outer layer 132, which can be an elastomeric material, over at least a portion of the tubular support 120 and up to a proximal end of the funnel shape 140 (see FIG. 4A). The proximal end of the funnel shape can be configured similarly to an example proximal end of the funnel shape 140 disclosed herein, variations thereof, and alternatives thereto as understood by a person skilled in the pertinent art. In some examples, the step of laminating the outer layer 132 can include laminating the outer layer 132 up to a distal end of the tubular support 120.

At block 405, the method 400 can include replacing the tapered mandrel 51 with a mandrel 52 pre-laminated with a layer of elastomeric material 151. The mandrel can be configured similarly to an example mandrel 52 disclosed herein, variations thereof, and alternatives thereto as understood by a person skilled in the pertinent art.

In some examples, the method 400 can include chemically etching the outer surface of the layer 151 and the outer surface of the outer layer 132.

At block 406, the method 400 can include laminating a third layer of polymer material 153 over the exposed outer surface 131a of the inner layer 131.

In some examples, the method 400 can further include terminating a distal end of the distal tip section 150 by trimming excess elastomeric material 151, 153.

In some examples, the outer layer 132 and the third layer 153 respectively have a thickness of at least 0.003″.

In some examples, the method 400 can further include laminating a fourth layer 155 of elastomeric material over some or all of the distal tip of the catheter 101.

FIG. 4B shows an alternate method of constructing the method 500 of manufacture for a clot capture catheter as disclosed herein. The method steps in FIG. 4B can be implemented by any of the example means described herein or by similar means, as will be appreciated.

At block 501, the method can include laminating a first layer (151) comprising elastomeric material over the larger outer diameter of a tapered mandrel (51).

In some examples, the mandrel (51) can be pre-coated with PTFE to allow easy removal of the tip from the mandrel.

At block 503, the method can include pulling a second layer (152/131) comprising polymer material up to and over the first layer (151) with the second layer (152/131) terminating at a position proximal of the distal end and distal of the proximal end of the first layer (151).

At block 505, the method can include positioning a tubular support (120) over the second layer (152) with the tip of the tubular support (120) positioned at the distal end of the second layer (152) and extending the tubular support (120) proximally of the distal end of the second layer (152).

At block 507, the method can include melting a third layer (153/132) over the tubular support (120) and extending distally past the tubular support (120), the first layer (151), and the second layer (152) by a pre-determined distance.

In some examples, the third layer can be extended distally past the tubular support between 0.25 mm and 0.75 mm. In this example, the first layer could terminate at the same location as the third layer or at a position between the distal end of the second layer and the proximal end of the third layer.

At block 509, the method can include removing the mandrel (52).

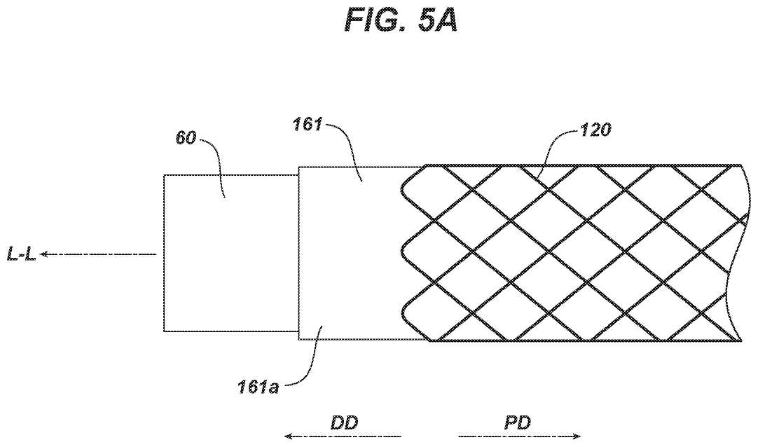

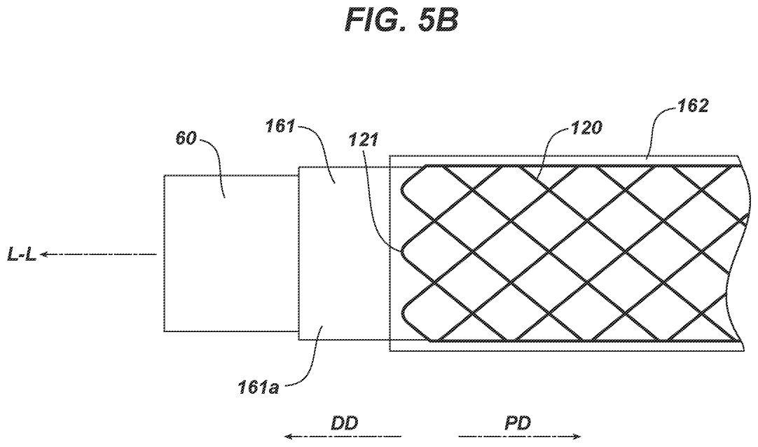

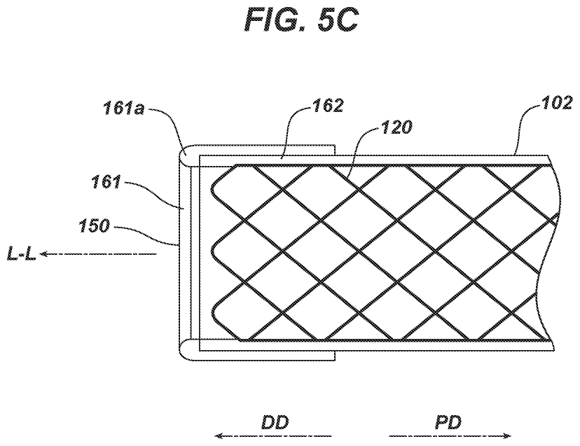

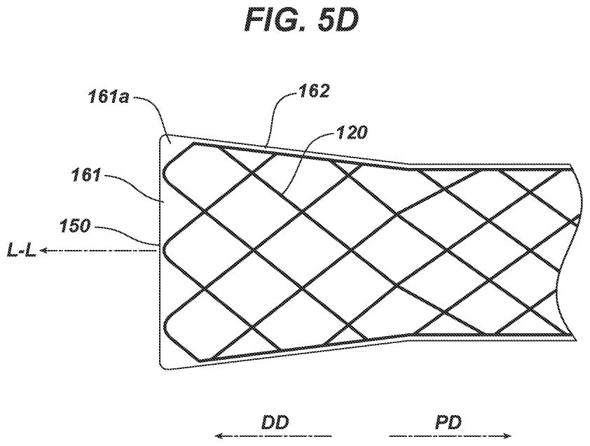

FIG. 5A is a side view illustrating a step of placing a tubular support 120 over the first layer 161, leaving a distal portion 161a of the first layer 161 exposed.

In some examples, the step shown in FIG. 5A can include loading the tubular support 120 and first layer 161 over a mandrel 60.

FIG. 5B is a side view illustrating a step of laminating the second layer 162 up to a distal end 121 of the tubular support 120, leaving the distal portion 161a of the first layer 161 exposed.

FIG. 5C is a side view illustrating a step of inverting the distal portion 161a of the first layer 161 proximally over at least a portion of the tubular support 120, covering the distal end of the distal tip section 150, and such that outer etched surface of the first layer 161 is now an inner etched surface of the first layer 162. Furthermore, the outer etched surface of the second layer 162 is in contact with the inner etched surface of the first layer 161 to form an efficient adhesion between the layers.

FIG. 5D is a side view illustrating a step of flaring a distal tip of the catheter 101 approximately a millimeter using a tapered mandrel 51.

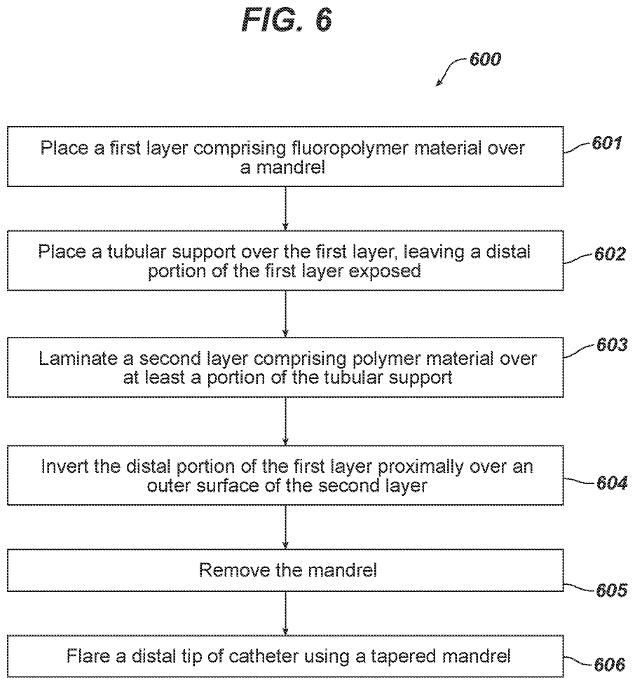

FIG. 6 shows a method 600 of manufacture for a clot capture catheter as disclosed herein. The method steps in FIG. 6 can be implemented by any of the example means described herein or by similar means, as will be appreciated.

At block 601, the method 600 can include placing a first layer 161 comprising fluoropolymer material over a mandrel 60. The mandrel can be configured similarly to an example mandrel 60 disclosed herein, variations thereof, and alternatives thereto as understood by a person skilled in the pertinent art.

In some examples, the method 600 can further include chemically etching an outer surface of the first layer 161.

At block 602, the method 600 can include placing a tubular support 120 over the first layer 161, leaving a distal portion of the first layer 161a exposed. The tubular support can be configured similarly to an example tubular support 120 disclosed herein, variations thereof, and alternatives thereto as understood by a person skilled in the pertinent art.

At block 603, the method 600 can include laminating a second layer 162 comprising elastomeric material over at least a portion of the tubular support 120. In some examples, laminating a second layer 162 over at least a portion of the tubular support 120 can include laminating the second layer 162 up to a distal end 121 of the tubular support 120, leaving the distal portion 161a of the first layer 161 exposed.

At block 604, the method 600 can include inverting the distal portion 161a of the first layer 161 proximally over an outer surface of the second layer 162 and laminating the layers together. In some examples, inverting the distal portion 161a of the first layer 161 can include inverting the distal portion 161a of the first layer 161 such that both the inner and outer surface of the second layer 162 are in contact with the outer surface of the first layer 161. This can be done to allow the chemically etched inverted section to bond with the outer surface of the second layer 162.

At block 605, the method 600 can include removing the mandrel 60. The mandrel can be configured similarly to an example mandrel 60 disclosed herein, variations thereof, and alternatives thereto as understood by a person skilled in the pertinent art.

At block 606, the method 600 can include flaring a distal tip of catheter 101 using a tapered mandrel 51. In some examples, heat will be needed to re-melt the elastomeric material. The heat required for this step is a temperature that will allow only the elastomeric materials to be melted and not the fluoropolymer materials. The distal tip of the catheter can be configured similarly to an example distal tip section 101 disclosed herein, variations thereof, and alternatives thereto as understood by a person skilled in the pertinent art. In some examples, flaring the distal tip of the catheter 101 using the tapered mandrel 51 can include flaring the distal tip of the catheter 101 approximately a millimeter.

In some examples, the second layer has a thickness of at least 0.003″.

Other aspects and features of the present disclosure will become apparent to those of skill in the pertinent art, upon reviewing the following detailed description in conjunction with the accompanying figures.

In describing examples, terminology is resorted to for the sake of clarity. It is intended that each term contemplates its broadest meaning as understood by those skilled in the pertinent art and includes all technical equivalents that operate in a similar manner to accomplish a similar purpose. It is also to be understood that the mention of one or more steps of a method does not preclude the presence of additional method steps or intervening method steps between those steps expressly identified. Steps of a method can be performed in a different order than those described herein without departing from the scope of the disclosed technology. Similarly, it is also to be understood that the mention of one or more components in a device or system does not preclude the presence of additional components or intervening components between those components expressly identified.

The descriptions contained herein are examples of the disclosure and are not intended in any way to limit the scope of the disclosure. While particular examples of the present disclosure are described, various modifications to devices and methods can be made without departing from the scope and spirit of the disclosure. For example, while the examples described herein refer to particular components, the disclosure includes other examples utilizing various combinations of components to achieve a described functionality, utilizing alternative materials to achieve a described functionality, combining components from the various examples, combining components from the various example with known components, etc. The disclosure contemplates substitutions of component parts illustrated herein with other well-known and commercially available products. To a person skilled in the pertinent art, these modifications are often apparent and are intended to be within the scope of the claims which follow.

What is claimed is:

1. A clot capture catheter comprising:
an elongate tubular shaft comprising a proximal end, a distal end, a lumen, a tubular support, an inner layer disposed within the tubular support, and an outer layer disposed over the tubular support; and
a distal tip section extending distally from the elongate tubular shaft, the distal tip section comprising a first layer and a third layer comprising an elastomeric material, and a second layer comprising a polymer material.

2. The catheter of claim 1, wherein the first layer and the third layer are joined at a distal end of the distal tip section.

3. The catheter of claim 1, wherein the tubular support comprises a braid.

4. The catheter of claim 1, wherein the first layer comprises a proximal terminate within the distal tip section.

5. The catheter of claim 1, wherein the distal tip section comprises a funnel shape such that a diameter of a distal end of the tip section is greater than a diameter of a proximal end of the tip section.

6. The catheter of claim 1, wherein the outer surface and at least a distal portion of the inner surface of the second layer is chemically etched.

7. The catheter of claim 1, wherein the third layer of the distal tip section and the outer layer of the elongate tubular shaft are contiguous.

8. A method for manufacturing a catheter, the method comprising:
placing an inner layer comprising an elastomeric material over a tapered mandrel;
placing a tubular support over the inner layer, leaving an exposed outer surface at a distal end of inner layer;
flaring a distal end of the inner layer into a funnel shape;
laminating an outer layer comprising elastomeric material over at least a portion of the tubular support and up to a proximal end of the funnel shape;
replacing the tapered mandrel with a mandrel pre-laminated with a first layer of elastomeric material; and
laminating a third layer of elastomeric material over the exposed outer surface of the inner layer.

9. The method of claim 8, further comprising:
terminating a distal end of the distal tip section by trimming excess elastomeric material.

10. The method of claim 8, further comprising:
chemically etching the outer surface of the first layer and the outer surface of the outer layer.

11. The method of claim 8, wherein the tapered mandrel is flared approximately a millimeter.

12. The method of claim 8, further comprising:
laminating the second layer up to a distal end of the tubular support.

13. The method of claim 8, wherein the second and third layers respectively have a thickness of at least 0.003″.

14. The method of claim 8, further comprising:
laminating a fourth layer of elastomeric material over some or all of the distal tip of the catheter.

15. A method for manufacturing a catheter, the method comprising:
placing a first layer comprising fluoropolymer material over a mandrel;
placing a tubular support over the first layer, leaving a distal portion of the first layer exposed;
laminating a second layer comprising elastomeric material over at least a portion of the tubular support;
inverting the distal portion of the first layer proximally over an outer surface of the second layer and laminating the layers together;
removing the mandrel; and
flaring a distal tip of catheter using a tapered mandrel.

16. The method of claim 15, further comprising:
chemically etching an outer surface of the first layer.

17. The method of claim 15, wherein inverting the distal portion of the first layer proximally over the outer surface of the second layer comprises inverting the distal portion of the first layer such that both an inner and outer surface of the second layer are in contact with the outer surface of the first layer.

18. The method of claim 15, wherein laminating the second layer over the tubular support comprises laminating the second layer up to a distal end of the tubular support, leaving the distal portion of the first layer exposed.

19. The method of claim 15, wherein the second layer has a thickness of at least 0.003″.

20. The method of claim 15, wherein flaring the distal tip of catheter using the tapered mandrel comprises flaring the distal tip of the catheter approximately a millimeter.
